(19) United States
(12) Patent Application Publication
(10) Pub. No.: US 2013/0108569 A1
(43) Pub. Date: May 2, 2013

(54) PIGMENT MIXTURE, AND USE THEREOF IN COSMETICS, FOOD AND PHARMACEUTICALS

(71) Applicant: Merck Patent Gesellschaft, Darmstadt (DE)

(72) Inventors: Veronika HOCHSTEIN, Bruchsal (DE); Christoph SCHMIDT, Kriftel (DE); Sabine SCHOEN, Herten (DE)

(73) Assignee: MERCK PATENT GESELLSCHAFT, DARMSTADT (DE)

(21) Appl. No.: 13/718,439

(22) Filed: Dec. 18, 2012

Related U.S. Application Data
(62) Division of application No. 12/300,012, filed on Nov. 7, 2008, now abandoned.

(30) Foreign Application Priority Data
May 9, 2006 (DE) 10 2006 021 784.5

(51) Int. Cl. C09C 1/00; A23L 1/275; A61K 8/29

(52) U.S. Cl. CPC C09C 1/0084; A61K 8/29; A23L 1/275
USPC 424/63; 426/250

(57) ABSTRACT

The invention relates to pigment mixtures comprising at least two components A and B. The inventive pigment mixtures are characterized in that component A is embodied as effect pigments while component B is embodied as coloring agents and fillers. Also disclosed is the use of said pigment mixtures in cosmetic formulations and for coloring food products and pharmaceutical products.

The present invention relates to effect pigments based on flake-form substrates, and to the use thereof in mixtures with other colorants and/or fillers in cosmetic formulations and in the foods and pharmaceuticals sector.

Gold pigments based on flake-form substrates are of importance, in particular, in printing applications and in cosmetics. Frequently, however, the gold pigments known from the prior art exhibit the disadvantage that they do not have a sufficiently intense colour and brightness and therefore do not cause a truly golden optical impression in the various application media.

The object of the present invention is to find gold pigments for cosmetics which are distinguished by a more intense golden lustre and do not have the above-mentioned disadvantages. A further object of the invention is to find formulations in which the golden lustre is supported in a particularly advantageous manner or modified in an optically attractive manner.

Surprisingly, it has now been found that pigment mixtures comprising effect pigments, preferably gold pigments, based on multicoated flake-form substrates in combination with further colorants and/or fillers impart a very soft skin feel, are light-stable, do not bleed/migrate, are non-toxic and have high hiding power. The pigments are distinguished by the fact that they have alternating high- and low-refractive-index layers and comprise at least one high-refractive-index coating consisting of a mixture of TiO₂ and Fe₂O₃ in the molar ratio 1:0.5 to 1:2.0.

Gold pigments based on multicoated flake-form substrates are disclosed, for example, in WO 01/30921.

The use of the multilayer pigment having a golden mass tone in combination with organic and inorganic fillers and/or with flake-form, needle-shaped, spherical or crystalline colorants enables colour effects to be enhanced and novel colour effects to be achieved. Furthermore, the pigment mixtures are distinguished by their high lustre and a very good skin feel.

The invention thus relates to a pigment mixture consisting of at least two components A and B, where
component A comprises effect pigments based on multicoated flake-form substrates which have a layer sequence comprising
   (A) a high-refractive-index coating consisting of a mixture of TiO₂ and Fe₂O₃ in the molar ratio 1:0.5 to 1:2.0 and optionally one or more metal oxide(s) in amounts of <20% by weight, based on layer (A),
   (B) a colourless coating having a refractive index n<1.8,
   (C) a colourless coating having a refractive index n>1.8,
   (D) an absorbent coating having a refractive index n>1.8
   and optionally
   (E) an outer protective layer,
   and
component B comprises colorants selected from the group of inorganic pigments, organic pigments, dyes, colouring natural fruit and/or plant extracts and/or fillers which consist of flake-form, needle-shaped, spherical or irregularly shaped particles.

The outstanding colouristic parameter of the pigment mixture is the strong, preferably purely golden, colour together with an optimum lustre effect and/or silk effect. The pigments of component A preferably have Lab values in the range from L=60 to 85; a=−15 to 25; b=22 to 45 (measurement method: Phyma, 22.5°/22.5° on a black background).

The invention likewise relates to cosmetic formulations, such as, for example, make-up, compact powders, loose powders, lipsticks, lotions, emulsions, etc., which comprise the pigment mixture according to the invention. The pigment mixtures are furthermore suitable for colouring foods and pharmaceutical products, including OTC preparations, and for colouring coatings of food and pharmaceutical products, including OTC preparations, such as, for example, medicament coatings of tablets, dragees, gelatine capsules, etc.

The effect pigments of component A can be mixed with the colorant or filler in any ratio. The mixing ratio of component A to component B is preferably 99:1 to 50:50, in particular 95:5 to 70:30, very particularly preferably 70:30 to 50:50. If component B comprises fillers, the ratio of component A:B may also be 99:1 to 1:99.

Preferred effect pigments of component A have the following structure;

substrate+Fe₂O₃/TiO₂+SiO₂+TiO₂+Fe₂O₃/TiO₂

substrate+Fe₂O₃/TiO₂+SiO₂+TiO₂+Fe₂O₃

substrate+Fe₂O₃/TiO₂+SiO₂+TiO₂+SiO₂+Fe₂O₃/TiO₂

Layers (A) to (D) or (A) to (E) are preferably applied directly to the substrate surface, i.e. layer (A) is located directly on the substrate surface.

In order to improve the pigment properties, such as lustre and brightness, interlayers (ILs) can optionally be applied, for example a layer of TiO₂, preferably with layer thicknesses of 1-100 nm, in particular 1-80 nm and very particularly preferably 1-50 nm, or a layer of SiO₂, preferably with layer thicknesses of 5-100 nm, can be applied to the substrate. In this case, particular preference is given to a layer structure comprising

substrate+(IL)TiO₂+Fe₂O₃/TiO₂+SiO₂+TiO₂+Fe₂O₃/TiO₂

substrate+(IL)TiO₂+Fe₂O₃/TiO₂+SiO₂+TiO₂+Fe₂O₃

The present invention likewise relates to these effect pigments having an interlayer and to the use thereof in paints, coatings, printing inks, plastics, in cosmetic formulations and for colouring food and pharmaceutical products.

Suitable base substrates for the effect pigments of component A are on the one hand opaque and on the other hand transparent flake-form substrates. Preferred substrates are phyllosilicates and glass flakes. Particularly suitable are natural and/or synthetic mica, talc, kaolin, flake-form iron or aluminium oxides, glass flakes, SiO₂ flakes, SiOₓ flakes (0.70≦x≦2.0), preferably SiO₂ flakes, TiO₂ flakes, graphite flakes, synthetic support-free flakes, liquid crystal polymers (LCPs), holographic pigments, BiOCl flakes, metal flakes, optionally passivated, such as, for example, aluminium flakes, flakes of aluminium bronzes, brass bronzes, zinc bronzes, titanium bronzes or other comparable materials.

The size of the base substrates is not crucial per se and can be matched to the particular application. In general, the flake-form substrates have a thickness between 0.02 and 5 μm, in particular between 0.05 and 4.5 μm. The size in the other two dimensions is usually between 1 and 250 μm, preferably between 2 and 200 μm, and in particular between 5 and 150 μm. Glass flakes preferably have a layer thickness of ≦1.0 μm, in particular ≦0.8 μm and very particularly preferably 0.5 μm.

The effect pigments have a high-refractive-index coating (A) consisting of a mixture of TiO₂ and Fe₂O₃, preferably in the molar ratio 1:1, in combination with a colourless low-refractive-index coating (B) alternating on the substrate. Layer (A) can be converted into pseudobrookite or a mixture of pseudobrookite with TiO₂ or pseudobrookite with Fe₂O₃ by suitable measures known to the person skilled in the art, such as, for example, calcination of the pigments at temperatures >800° C.

Layer (C) preferably consists of TiO₂, ZrO₂, SnO₂, Ce₂O₃, BiOCl or mixtures or combinations thereof. In the case where layer (C) consists of TiO₂, the TiO₂ is preferably in the rutile modification.

Suitable materials for layer (D) are absorbent materials, such as metals, for example iron, tungsten, chromium, cobalt, nickel, copper, silver, gold, aluminium and alloys thereof, metal oxides, such as, for example, CoO, CoO₃O₄, Fe₂O₃, Fe₃O₄, pseudobrookite, TiO₂/Fe₂O₃ mixture, VO₂, V₂O₃, metal sulfides, such as, for example, molybdenum sulfide, iron sulfide, tungsten sulfide, chromium sulfide, cobalt sulfide, nickel sulfide and mixtures of these sulfides. The absorbent layer (D) is preferably a mixture of TiO₂ and Fe₂O₃, where the mixing ratios, like in the case of layer (A), can be varied in broad limits. The TiO₂ to Fe₂O₃ molar ratio is preferably 1:1. Layer (D) can be converted into pseudobrookite or a mixture of pseudobrookite with TiO₂ or pseudobrookite with Fe₂O₃ by suitable measures analogously to layer (A), such as, for example, calcination of the pigments at temperatures >800° C. This layer (D) has a refractive index of n>1.8, in particular n≧2.0.

A further colourless low-refractive-index layer (B*), which may be identical to or different from layer (B), may be located between layers (C) and (D).

The high-refractive-index layer (A) preferably has a refractive index of n>1.8, in particular n≧2.0, and is a mixture of TiO₂ and Fe₂O₃, where the mixing ratio is 1:0.5 to 1:2.0, preferably 1:0.7 to 1:1.5, in particular 1:1. Layer (A) is preferably intensely coloured pseudobrookite. The thickness of layer (A) is preferably 10 to 300 nm, preferably 15 to 250 nm and in particular 20 to 200 nm.

In order to increase the tinting strength of layer (A), it is also advisable to admix one or more metal oxides from the group Al₂O₃, Ce₂O₃, B₂O₃, ZrO₂, SnO₂. The % by weight proportion of the further metal oxides, besides the Fe₂O₃/TiO₂ mixture, should be not greater than 20% by weight, preferably not greater than 10% by weight.

In the case where layer (D) is likewise a layer of a TiO₂/Fe₂O₃ mixture, it is likewise advisable to add one or more metal oxides, such as, for example, Al₂O₃, Ce₂O₃, B₂O₃, ZrO₂, SnO₂, in amounts of not greater than 20% by weight, based on layer (D), in order to increase the tinting strength.

Suitable colourless low-refractive-index materials which are suitable for coating (B) are preferably metal oxides or the corresponding oxide hydrates, such as, for example, SiO₂, Al₂O₃, AlO(OH), B₂O₃, MgF₂, MgSiO₃ or a mixture of the said metal oxides. The thickness of layer (B) is 10 to 600 nm, preferably 20 to 500 nm and in particular 20 to 400 nm.

If layer (C) is a TiO₂ layer, this is preferably in the rutile modification. The processes for the preparation of rutile are described in the prior art, for example in U.S. Pat. No. 5,433,779, U.S. Pat. No. 4,038,099, U.S. Pat. No. 6,626,989, DE 25 22 572 C2, EP 0 271 767 B1. Before the precipitation of TiO₂ onto layer (B), a thin layer of tin oxide is preferably applied (layer B*), which serves as additive for conversion of the TiO₂ into rutile.

The effect pigments can be prepared, for example, as described in WO 01/30921.

The coating of the substrate flakes with layers (A)-(D) can be carried out by wet-chemical methods and/or by CVD methods. Before the application of layer (A), a thin dielectric layer where n<1.8 can optionally also be deposited. A coating of this type, for example on glass flakes, can consist, for example, of an SiO₂ layer with a thickness of 5-100 nm.

The effect pigments are preferably prepared using the wet-chemical method, it being possible to use the known wet-chemical coating technologies which were developed for the preparation of pearlescent pigments and are described, for example, in the following publications: DE 14 67 468, DE 19 59 988, DE 20 09 566, DE 22 14 545, DE 22 15 191, DE 22 44 298, DE 23 13 331, DE 25 22 572, DE 31 37 808, DE 31 37 809, DE 31 51 343, DE 31 51 354, DE 31 51 355, DE 32 11 602, DE 32 35 017.

In the case of wet coating, the substrate particles are suspended in water, and one or more hydrolysable metal salts are added at a suitable pH for the hydrolysis, which is selected so that the metal oxides or metal oxide hydrates are precipitated directly onto the flakes without significant secondary precipitations occurring. During the coating operation, the substrate particles are kept in motion in order that a homogeneous coating of the substrate particles is ensured and the substrate is completely enveloped and no open edges remain. The pH is usually kept constant by simultaneous metered addition of a base and/or acid. The pigments are subsequently separated off, washed and preferably dried at 50-180° C. and optionally calcined, where the calcination temperature must be optimised with respect to the coating present in each case and the substrate used. In general, the calcination temperatures are between 250 and 1000° C., preferably between 350 and 900° C. If desired, the pigments can be separated off after application of individual coatings, dried and optionally calcined and then re-suspended for the precipitation of further layers.

Furthermore, the coating can also be carried out in a fluidised-bed reactor by gas-phase coating, where the processes proposed, for example, in EP 0 045 851 A1 and EP 0 106 235 A1 for the preparation of pearlescent pigments can be used correspondingly. It is necessary here for the substrate to be kept uniformly in motion during the coating operation in order that homogeneous coating of all particle surfaces is ensured and the substrate is completely enveloped and no open edges remain.

The effect pigments of component A can also be provided with an organic or inorganic protective layer (layer E) in order to improve the light, weather and chemical stability or in order to increase the compatibility in various media. Suitable post-coatings or post-treatments are, for example, silanes, silicones, adsorbent silicones, metal soaps, amino acids, lecithins, fluorine components, polyethylenes, collagen or the methods described in DE 22 15 191, DE 31 51 354, DE 32 35 017 or DE 33 34 598, EP 0 632 109, U.S. Pat. No. 5,759,255, DE 43 17 019, DE 39 29 423, EP 0 492 223, EP 0 342 533, EP 0 268 918, EP 0 141 174, EP 0 764 191, WO 98/13426 or EP 0 465 805. This post-coating further increases the chemical and photochemical stability or simplifies handling of the gold pigment, in particular incorporation into various media. In order to improve the wettability, dispersibility and/or compatibility with the user media, it is possible to apply, for example, functional coatings of Al₂O₃ or ZrO₂ or mixtures or mixed phases thereof to the pigment surface. Organic or combined organic/inorganic post-coatings, for example with silanes, as described, for example, in EP 0090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff. and P. H. Harding J. C. Berg, J. Adhesion Sci. Technol. Vol. 11 No. 4, pp. 471-493, are furthermore possible. The additionally applied substances make up only about 0.1 to 5% by weight, preferably 0.5 to 3.0% by weight, of the entire pigment.

The post-coating of the effect pigments can be carried out directly in a one-pot process onto layer (D). However, it is also possible firstly to isolate, optionally dry and calcine the multilayer pigment and subsequently to apply the post-coating.

Besides the effect pigment, preferably a gold pigment (component A), pigment mixtures according to the invention comprise a filler and/or a colorant of component B.

Suitable as component B for the pigment mixture according to the invention are all flake-form, needle-shaped, spherical and crystalline colorants or fillers which are known to the person skilled in the art, in particular those which have a particle size of 0.001 to 10 μm, preferably 0.01 to 1 μm. Colorants are taken to mean inorganic and organic colorants in accordance with DIN standard 55944. In this application, colorants are also taken to mean colouring natural fruit and plant extracts.

The pigment mixtures according to the invention preferably comprise, as colorants, inorganic dyes and inorganic pigments, such as, for example, inorganic white pigments, inorganic coloured pigments, inorganic black pigments, inorganic effect pigments. The latter are of course not identical with the pigments of component A. Besides the inorganic colorants, organic colorants, such as, for example, organic pigments, such as, for example, coloured pigments, black pigments, effect pigments, and inorganic dyes, such as, for example, coloured dyes, black dyes, are also suitable. Suitable fillers are preferably flake-form or spherical materials.

Component B preferably comprises coated or uncoated SiO₂ beads. SiO₂ beads coated with one or more metal oxides are disclosed, for example, in EP 0 803 550 A2.

The colorants of component B are furthermore preferably inorganic effect pigments, such as, for example, pearlescent pigments, including multilayer pigments or interference pigments, which are not identical with component (A). The pearlescent pigments used are pigments based on flake-form, transparent or semitransparent substrates comprising, for example, phyllosilicates, such as, for example, natural or synthetic mica, talc, sericite, kaolin or other silicate materials, coated with coloured or colourless metal oxides, such as, for example, TiO₂, titanium suboxides, titanium oxynitrides, Fe₂O₃, Fe₃O₄, FeOOH, SnO₂, Cr₂O₃, ZnO, CuO, NiO, and other metal oxides, alone or in a mixture, in a single layer or in successive layers.

Pearlescent pigments are disclosed, for example, in German patents and patent applications 14 67 468, 19 59 998, 20 09 566, 22 14 454, 22 15 191, 22 44 298, 23 13 331, 25 22 572, 31 37 808, 31 37 809, 31 51 343, 31 51 354, 31 51 355, 32 11 602, 32 35 017 and P 38 42 330 and are commercially available, for example under the trademarks Iriodin®, Timiron®, Xirona® from Merck KGaA, Darmstadt, Germany and/or Rona, USA. Particularly preferred pigment compositions comprise TiO₂/mica, Fe₂O₃/mica and/or TiO₂/Fe₂O₃/mica pigments. The pearlescent pigments may additionally have a layer of Berlin Blue or Carmine Red on the surface.

Preference is furthermore given to coated or uncoated BiOCl pigments, TiO₂- and/or Fe₂O₃-coated SiO₂, glass or Al₂O₃ flakes. The coating of the SiO₂ flakes with one or more metal oxides can be carried out, for example, as described in WO 93/08237 (wet-chemical coating) or DE-A 196 14 637 (CVD method).

The multilayer pigments disclosed, for example, in DE-A 196 18 563, DE-A 196 18 566, DE-A 196 18 569, DE-A 197 07 805, DE-A 197 07 806, DE-A 197 46 067 are based on a flake-form, transparent, coloured or colourless matrix consisting of mica (synthetic or natural), SiO₂ flakes, glass flakes, Al₂O₃ flakes, polymer flakes and generally have a thickness between 0.3 and 5 μm, in particular between 0.4 and 2.0 μm. The size in the other two dimensions is usually between 1 and 250 μm, preferably between 2 and 100 μm, and in particular between 5 and 40 μm. The multilayer pigments consist of the matrix (substrate) coated with metal oxides (at least 2). The coating of the substrate flakes mica, SiO₂ flakes, glass flakes, Al₂O₃ flakes with a plurality of layers is carried out in such a way that a layer structure preferably consisting of alternating high- and low-refractive-index layers is formed. The multilayer pigments preferably contain 2, 3, 4, 5, 6 or 7 layers, in particular 3, 4 or 5 layers. Suitable high-refractive-index metal oxides are, for example, titanium dioxide, zirconium oxide, zinc oxide, iron oxides, iron/titanium oxides (iron titanates) and/or chromium oxide, in particular TiO₂ and/or Fe₂O₃. The low-refractive-index oxides used are SiO₂ and Al₂O₃. However, it is also possible to employ MgF₂ or an organic polymer (for example acrylate) for this purpose. The coating of the substrate flakes can be carried out, for example, as described in WO 93/08237 (wet-chemical coating) or DE-A-196 14 637 (CVD method). Particularly preferred multilayer pigments based on mica (natural or synthetic), glass flakes, Al₂O₃ flakes, Fe₂O₃ flakes, SiO₂ flakes comprise a layer sequence TiO₂—SiO₂—TiO₂. The TiO₂ can be in either the anatase or the rutile modification. It is preferably in the form of rutile.

The interference pigments are preferably pigments based on mica, glass flakes, SiO₂ flakes which are coated with coloured or colourless metal oxides, such as, for example, TiO₂, titanium suboxides, titanium oxynitrides, Fe₂O₃, Fe₃O₄, SnO₂, Cr₂O₃, ZnO, CuO, NiO, and other metal oxides, alone or in a mixture, in a single layer or in successive layers.

Suitable flake-form colorants are, in particular, pearlescent pigments, in particular based on mica, SiO₂ flakes or Al₂O₃ flakes, which are only covered with one metal-oxide layer, metal-effect pigments (Al flakes, bronzes), optically variable pigments (OVPs), liquid-crystal polymer pigments (LCPs) or holographic pigments.

The spherical colorants include, in particular, TiO₂, coloured SiO₂, CaSO₄, iron oxides, chromium oxides, carbon black, organic coloured pigments, such as, for example, anthraquinone pigments, quinacridone pigments, diketopyrrolopyrrole pigments, phthalocyanine pigments, azo pigments, isoindoline pigments. The needle-shaped pigments are preferably BiOCl, coloured glass fibres, α-FeOOH, organic coloured pigments, such as, for example, azo pigments, β-phthalocyanine CI Blue 15.3, Cromophtal Yellow 8GN (Ciba), Irgalith Blue PD56 (Ciba), azomethine copper complex CI Yellow 129, Irgazine Yellow 5GT (Ciba).

It is likewise possible to admix nanoscale dielectrics in order to improve the skin feel. Examples of admixtures of this type are Al₂O₃, SiO₂, ZnO or TiO₂, which are usually added to the formulation in amounts of 0.01-15%.

The pigment mixture according to the invention is simple and easy to handle. The pigment mixture can be incorporated into the application system by simple stirring-in. Components A and B can be added to the application system simultaneously, successively or as a mixture. Complex grinding and dispersion of the pigments is unnecessary.

The pigment mixture according to the invention can preferably be used for pigmenting food colourings, for the finishing of foods, for example mass colouring or as a coating, in medicament coatings, for example in dragees and tablets, or in cosmetic formulations, such as lipsticks, lip gloss, eyeliner, eye shadow, rouge, sunscreen, pre-sun and after-sun compositions, make-ups, body lotions, bath gels, soaps, bath salts, toothpaste, hair gels, (volume) mascara, nail varnishes, compact powders, shampoos, loose powders and gels, etc.

The concentration of the pigment mixture in the application system to be pigmented is generally between 0.01 and 70% by weight, preferably between 0.1 and 50% by weight and in particular between 1.0 and 10% by weight, based on the total solids content of the system. It is generally dependent on the specific application and can be up to 100% in the case of loose powders. The use concentration of the pigment mixture according to the invention extends from 0.01% by weight in shampoos to 70% by weight in compact powders. In a mixture of the multilayer pigments of component A with spherical fillers, for example SiO₂, the concentration can be 0.01-70% by weight in the formulation. The cosmetic products, such as, for example, nail varnishes, lipsticks, compact powders, shampoos, loose powders and gels, are distinguished by particularly interesting lustre effects.

The pigment mixture according to the invention can advantageously be employed in both decorative and care cosmetics. The use concentration and the mixing ratio of the multilayer pigments of component A with component B, in particular organic and inorganic coloured pigments and dyes, of natural or synthetic origin, such as, for example, chromium oxide, ultramarine, spherical SiO₂ or TiO₂ pigments, are dependent on the application medium and the effect to be achieved.

The effect pigment of component A can furthermore be mixed with commercially available fillers. Fillers which may be mentioned are, for example, natural and synthetic mica, glass beads or glass powder, nylon powder, pure or filled melamine resins, talc, glasses, kaolin, oxides or hydroxides of aluminium, magnesium, calcium or zinc, BiOCl, barium sulfate, calcium sulfate, calcium carbonate, magnesium carbonate, carbon, and physical or chemical combinations of these substances.

There are no restrictions regarding the particle shape of the filler. In accordance with requirements, it can be, for example, flake-form, spherical, needle-shaped, crystalline or amorphous.

The pigment mixture according to the invention can of course also be combined in the formulations with cosmetic raw materials and assistants of any type. These include, inter alia, oils, fats, waxes, film formers, surfactants, antioxidants, such as, for example, vitamin C or vitamin E, stabilisers, odour enhancers, silicone oils, emulsifiers, solvents, such as, for example, ethanol or ethyl acetate or butyl acetate, preservatives and assistants which generally determine applicational properties, such as, for example, thickeners and rheological additives, such as, for example, bentonites, hectorites, silicon dioxides, Ca silicates, gelatines, high-molecular-weight carbohydrates and/or surface-active assistants, etc.

The formulations comprising the pigment mixtures according to the invention can belong to the lipophilic, hydrophilic or hydrophobic type. In the case of heterogeneous formulations having discrete aqueous and non-aqueous phases, the pigment mixtures according to the invention may in each case be present in only one of the two phases or alternatively distributed over both phases.

The pH of the formulations can be between 1 and 14, preferably between 2 and 11 and particularly preferably between 5 and 8.

No limits are set for the concentrations of the pigment mixtures according to the invention in the formulation. They can be—depending on the application—between 0.001 (rinse-off products, for example shower gels) and 100% (for example lustre-effect articles for particular applications).

The pigment mixture according to the invention can furthermore also be combined with cosmetic active compounds. Suitable active compounds are, for example, insect repellents, inorganic UV filters, such as, for example, TiO₂, UV A/BC protective filters (for example OMC, B3, MBC), also in encapsulated form, anti-ageing active compounds, vitamins and derivatives thereof (for example vitamin A, C, E, etc.), self-tanning agents (for example DHA, erythrulose, inter alia), and further cosmetic active compounds, such as, for example, bisabolol, LPO, VTA, ectoine, emblica, allantoin, bioflavonoids and derivatives thereof. Organic UV filters are generally incorporated into cosmetic formulations in an amount of 0.5 to 10% by weight, preferably 1-8% by weight, and inorganic filters in an amount of 0.1 to 30% by weight, based on the formulation as a whole.

The compositions according to the invention may in addition comprise further conventional skin-protecting or skin-care active compounds. These may in principle be any active compounds known to the person skilled in the art.

Particularly preferred active compounds are pyrimidinecarboxylic acids and/or aryl oximes.

Of the cosmetic applications, particular mention should be made of the use of ectoine and ectoine derivatives for the care of aged, dry or irritated skin. Thus, EP-A-0 671 161 describes, in particular, that ectoine and hydroxy-ectoine are employed in cosmetic compositions, such as powders, soaps, surfactant-containing cleansing products, lipsticks, rouge, make-up, care creams and sunscreen preparations. The cosmetic formulations according to the invention preferably comprise 0.05-5% by weight, in particular 0.1-3% by weight, of ectoine or ectoine derivatives, based on the formulation.

Application forms of the cosmetic formulations which may be mentioned are, for example: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower preparations. Any desired customary vehicles, assistants and, if desired, further active compounds may be added to the composition.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic compositions may exist in various forms. Thus, they can be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Further embodiments are oily lotions based on natural or synthetic oils and waxes, lanolin, fatty acid esters, in particular triglycerides of fatty acids, or oily/alcoholic lotions based on a lower alcohol, such as ethanol, or a glycerol, such as propylene glycol, and/or a polyol, such as glycerol, and oils, waxes and fatty acid esters, such as triglycerides of fatty acids.

Solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling and treating the hair, in the form of a lotion or gel for brushing or blow-waving, in the form of a hair lacquer, permanent waving composition, colorant or bleach for the hair. The composition having light-protection properties may comprise adjuvants, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The pharmaceutical and food products are coloured by adding the pigment mixture according to the invention, preferably comprising at least one gold pigment (component (A)) and a colorant from the area of the natural or nature-identical dyes (component (B)), in the desired mixing ratios, to the product to be coloured in amounts of 0.005 to 15% by weight, preferably 0.01 to 100% by weight.

The admixing of natural or nature-identical dyes, organic or inorganic coloured pigments (component (B)) or colouring natural fruit and plant extracts approved for the foods sector enables the colour effect of the gold pigment in the product to be influenced and at the same time enables novel iridescent colour effects to be achieved.

Suitable natural or nature-identical dyes for the pigment mixture according to the invention are, in particular, E 101, E 104, E 110, E 124, E 131, E 132, E 140, E 141, E 151, E 160a. Suitable coloured pigments for the pigment mixture according to the invention are, for example, E 171, E 172, E 153.

The proportion of dyes besides the gold pigment, based on the food or pharmaceutical product, is preferably in the range from 0.5 to 25% by weight. The dye employed can likewise be fruit and plant extracts, such as, for example, carrot juice, beetroot juice, elderberry juice, hibiscus juice, paprika extract, aronia extract.

The total concentration of all pigments in the product to be pigmented should not exceed 50% by weight, based on the product. It is generally dependent on the specific application.

Various active-compound additives, such as, for example, vitamins, enzymes, trace elements, proteins, carbohydrates, essential fats and/or minerals, can also be added to the food and pharmaceutical products, where the total amount of active compounds, based on the food or pharmaceutical product, should not exceed 25% by weight. The amount of active compounds or active-compound mixtures is preferably 0.01-20% by weight, based on the product.

The products are coloured by adding the pigment mixture according to the invention, alone or in combination with assistants, cosmetic active compounds, ingredients, etc., to the product to be coloured, directly or in the presence of water and/or an organic solvent, in the desired mixing ratios, simultaneously or successively, during or after production thereof, before or after shaping (for example during extrusion, pelleting, expansion, granulation, etc.). Admixing of the effect pigments with pulverulent or loose powders is likewise possible.

The pigment mixture according to the invention can also be applied to the surface for colouring food and pharmaceutical products after shaping. In this case, the pigment mixture according to the invention is generally mixed with an application medium and subsequently applied to the product using suitable application and spray devices. The application or coating medium then ensures corresponding adhesion of the pigment mixture to the product surface. The latter is then coloured correspondingly.

On incorporation into the product matrix itself, the amount of the pigment mixture according to the invention used is preferably 0.5-40% by weight, in particular 1-30% by weight. In the case of surface colouring of food and pharmaceutical products, the use range in the colouring or coating solution used is 0.1-25% by weight, in particular 1-15% by weight. On use of the pigment mixture according to the invention in pulverulent products, the use range is 0.05-50% by weight, in particular 2-10% by weight.

The coating solutions preferably comprise water or organic solvents, such as, for example, ethanol or isopropanol. The film former employed in the coating solutions is preferably a cellulose derivative, such as, for example, hydroxypropylmethylcellulose. Particular preference is given to application solutions comprising cellulose derivatives which, instead of water, comprise 5-80% by weight of a suitable organic solvent.

Compared with aqueous coating solutions, the alcoholic or alcoholic-aqueous, cellulose-containing application solutions have significant applicational advantages:
use of cooler drying air during the spray application
colouring of heat-sensitive products, such as, for example, vitamin-containing foods, with the gold pigments is very readily possible.

Products which are suitable for colouring that may be mentioned are, in particular, coatings on all types of foods, in particular pigmented sugar and shellac coatings (alcoholic and aqueous), coatings with oils and waxes, with gum arabic and with cellulose grades (for example HPMC=hydroxypropyl-methylcellulose), with starch and albumin derivatives, carrageenan and other substances known to the person skilled in the art which are suitable for coating. The pigment mixture according to the invention is generally mixed with the application medium here and subsequently applied to the food or pharmaceutical product using suitable application and spray devices, or by hand. The application or coating medium then ensures corresponding adhesion of the pigments to the food or pharmaceutical product surface.

The latter is then coloured correspondingly. The application and coating solutions preferably comprise 0.1-20% by weight, in particular 2-15% by weight, of pigment mixture.

Preferred dry powder mixtures for coatings comprise a cellulose derivative, such as, for example, hydroxypropylmethylmethycellulose, sodium carboxymethylcellulose, a release agent, such as, for example, lecithin or stearic acid, a lustre enhancer, such as, for example, maltodextrin and/or dextrose, and the pigment mixture according to the invention. Dry powder mixtures of this type preferably comprise the pigment mixture in amounts of 0.01-50% by weight, in particular 0.5-40% by weight, based on the powder mixture. If necessary, dyes, flavourings, vitamins, sweeteners, etc., can also be added to these dry powder mixtures.

Products which are suitable for colouring or coating are, for example, sugar products, cake decorations, compresses, dragees, chewing gum, gum products, fondant products, marzipan products, filling compositions, cocoa and fat glazes, chocolate and chocolate-containing products, ice cream, cereals, snack products, coating compositions, cake glazes, scattered sugar decorations, nonpareils, jelly and gelatine products, sweets, liquorice, icing, candyfloss, fat, sugar and cream compositions, blancmange, desserts, flan glaze, cold fruit soups, soft drinks and carbonated beverages, beverages with stabilising additives, such as, for example, carboxymethylcellulose, acidified and unacidified milk products, such as, for example, quark, yoghurt, cheese, cheese rinds, sausage casings, etc.

In the case of coated food and pharmaceutical products, it is possible to combine the pigment mixture according to the invention with aroma substances (powder or liquid aromas), acids and/or with sweeteners, such as, for example, aspartame, in order additionally to reinforce the visual effect in terms of flavour.

The invention thus relates to all formulations from the foods and pharmaceuticals sector comprising the effect pigment of component A with further pigments, fillers or dyes (natural or nature-identical), colouring natural fruit and plant extracts, as colorants of component B.

A further major area of application is in the pharmaceuticals and OTC sector for colouring or as a coating for tablets, gelatine capsules, dragees, ointments, cough mixture, etc. In combination with conventional coatings, such as polymethacrylates and cellulose grades, for example HPMC, the pigment mixture according to the invention or the gold pigment can be employed in a variety of ways for colouring and finishing the products.

The invention thus also relates to formulations comprising the pigment mixture according to the invention.

The invention likewise relates to formulations comprising the pigment mixture according to the invention comprising components A and B in combination with water, polyols, polar and non-polar oils, fats, waxes, film formers, polymers, copolymers, surfactants, free-radical scavengers, antioxidants, stabilisers, odour enhancers, silicone oils, emulsifiers, solvents, preservatives, thickeners, rheological additives, fragrances, UV absorbers, surface-active assistants and/or cosmetic active compounds.

The following examples are intended to explain the invention, but without limiting it.

### EXAMPLES

### Example 1

100 g of mica having a particle size of 10-60 μm are heated to 75° C. in 2 l of demineralised water. When this temperature has been reached, a mixed solution of 157.5 g of TiCl₄ (30% by weight of TiCl₄), 236.6 g of FeCl₃ solution (11.7% of Fe), 5.9 g of AlCl₃×6H₂O and 60 g of demineralised water is slowly metered in with vigorous stirring. The pH is kept constant at pH 2.6 using 32% sodium hydroxide solution. When this solution has been added, the mixture is stirred for about a further 15 minutes. The pH is subsequently raised to pH=7.5 using 32% sodium hydroxide solution, and 431 g of sodium water-glass solution (13.5% of SiO₂) are slowly metered in at this pH. The pH is then lowered to 2.0 using 10% hydrochloric acid, the mixture is stirred for a further 15 minutes, and 393 g of TiCl₄ solution (370 g of TiCl₄/I) are metered in. During this addition, the pH is kept constant using 32% sodium hydroxide solution. The pH is subsequently raised to 2.9 using 32% sodium hydroxide solution, and a solution consisting of 34 g of FeCl₃×6H₂O and 49 g of demineralised water is slowly metered in. The pH is kept constant at pH=2.9 using 32% sodium hydroxide solution. When this solution has been added, the mixture is stirred for about a further 15 minutes. The pH is subsequently raised to pH=5.0 using 32% sodium hydroxide solution, and the mixture is stirred for a further 15 minutes.

The pigment is filtered off, washed with demineralised water and dried at 110° C. for 16 h. Finally, the pigment is calcined at 850° C. for 30 minutes, giving a gold pigment having an intense colour, high hiding power and strong lustre.

The intensely lustrous gold pigment has the following L, a, b values: (Phyma 22.50/22.5°, black background):

L=77.7; a=1.4; b=32.2

### Example 2

100 g of mica having a particle size of 10-60 μm are heated to 75° C. in 2 l of demineralised water. When this temperature has been reached, a mixed solution of 215 g of FeCl₃×6H₂O (11.7% of Fe), 144 g of TiCl₄ solution (30% by weight of TiCl₄) and 5.4 g of AlCl₃×6H₂O and 50 g of demineralised water is slowly metered in with vigorous stirring. The pH is kept constant at pH 2.6 using 32% sodium hydroxide solution. When this solution has been added, the mixture is stirred for about a further 15 minutes. The pH is subsequently raised to pH=7.5 using 32% sodium hydroxide solution, and 394 g of sodium water-glass solution (13.5% of SiO₂) are slowly metered in at this pH. The pH is then lowered to 2.0 using 10% hydrochloric acid. The mixture is stirred for a further 15 minutes, and 314 g of TiCl₄ solution (370 g of TiCl₄/I) are then metered in. During this addition, the pH is kept constant using 32% sodium hydroxide solution. The pH is subsequently raised to 2.9 using 32% sodium hydroxide solution, and a solution consisting of 86 g of FeCl₃×6H₂O solution (11.7% of Fe) and 50 g of demineralised water is slowly metered in. The pH is kept constant at 2.9 using 32% sodium hydroxide solution. When this solution has been added, the mixture is stirred for about a further 15 minutes. The pH is subsequently raised to pH=5.0 using 32% sodium hydroxide solution, and the mixture is stirred for a further 15 minutes.

The pigment is filtered off, washed with demineralised water and dried at 110° C. for 16 h. Finally, the pigment is calcined at 850° C. for 30 minutes, giving a gold pigment having an intense colour, high hiding power and strong lustre.

The intensely lustrous gold pigment has the following L, a, b values: (Phyma 22.50/22.50, black background):

L=80.0; a=−0.9; b=34.0

### Example 3

100 g of mica having a particle size of 10-60 μm are heated to 75° C. in 2 l of demineralised water. When this temperature has been reached, a mixed solution of 215 g of FeCl₃×6H₂O solution (11.7% of Fe), 144 g of TiCl₄ solution (30% by weight of TiCl₄) and 5.4 g of AlCl₃×6H₂O in 50 g of demineralised water is slowly metered in with vigorous stirring. The pH is kept constant at pH 2.6 using 32% sodium hydroxide solution. When this solution has been added, the mixture is stirred for about a further 15 minutes. The pH is subsequently raised to pH=7.5 using 32% sodium hydroxide solution, and 394 g of sodium water-glass solution (13.5% of SiO₂) are slowly metered in at this pH. The pH is then lowered to 2.0 using 10% hydrochloric acid, the mixture is stirred for a further 15 minutes, and 235 g of TiCl₄ solution (370 g of TiCl₄/I) are metered in. During this addition, the pH is kept constant using 32% sodium hydroxide solution. 22 g of sodium water-glass solution (13.5% of SiO₂) are subsequently slowly metered in at pH=2.0, and the mixture is stirred for a further 15 minutes. The pH is then raised to 2.6 using 32% sodium hydroxide solution, the mixture is stirred for a further minutes, and a mixed solution consisting of 116 g of FeCl₃×6H₂O (11.7% of Fe), 78 g of TiCl₄ solution (30% by weight of TiCl₄) and 2.5 g of AlCl₃×6H₂O and 30 g of demineralised water is slowly metered in. The pH is kept constant at pH=2.6 using 32% sodium hydroxide solution. When this solution has been added, the mixture is stirred for about a further 15 minutes. The pH is subsequently raised to pH=5.0 using 32% sodium hydroxide solution, and the mixture is stirred for a further 15 minutes. The pigment is filtered off, washed with demineralised water and dried at 110° C. for 16 h. Finally, the pigment is calcined at 850° C. for 30 minutes, giving a gold pigment having an intense colour, high hiding power and strong lustre.

The intensely lustrous gold pigment has the following L, a, b values: (Phyma 22.5°/22.5°, black background):

L=75.0; a=−1.2; b=28.3

### Example 4

100 g of mica having a particle size of 10-60 μm are heated to 75° C. in 2 l of demineralised water. When this temperature has been reached, a mixed solution of 215 g of FeCl₃×6H₂O solution (11.7% of Fe), 144 g of TiCl₄ solution (30% by weight of TiCl₄) and 5.4 g of AlCl₃×6H₂O and 50 g of demineralised water is slowly metered in with vigorous stirring. The pH is kept constant at pH 2.6 using 32% sodium hydroxide solution. When this solution has been added, the mixture is stirred for about a further 15 minutes. The pH is subsequently raised to pH=7.5 using 32% sodium hydroxide solution, and 394 g of sodium water-glass solution (13.5% of SiO₂) are slowly metered in at this pH. The pH is then lowered to 2.0 using 10% hydrochloric acid, the mixture is stirred for a further 15 minutes, and a solution of 3.0 g of SnCl₄×5H₂O and 10 ml of hydrochloric acid (37% of HCl) in 100 ml of deionised water is added at this pH. The pH is then lowered to 1.8 using 10% hydrochloric acid, the mixture is stirred for a further 15 minutes, and 235 g of TiCl₄ solution (370 g of TiCl₄/I) are metered in. During this addition, the pH is kept constant using 32% sodium hydroxide solution. The pH is then raised to 2.6 using 32% sodium hydroxide solution, the mixture is stirred for a further 15 minutes, and a mixed solution of 116 g of FeCl₃×6H₂O solution (11.7% of Fe), 78 g of TiCl₄ solution (30% by weight of TiCl₄) and 2.5 g of AlCl₃×6H₂O and 30 g of demineralised water is slowly metered in. The pH is kept constant at pH=2.6 using 32% sodium hydroxide solution. After the metal-salt solution has been added, the mixture is stirred for about a further 15 minutes. The pH is subsequently raised to pH=5.0 using 32% sodium hydroxide solution, and the mixture is stirred for a further 15 minutes.

The pigment is filtered off, washed with demineralised water and dried at 110° C. for 16 h. Finally, the pigment is calcined at 850° C. for 30 minutes, giving an intensely lustrous gold pigment having the following L, a, b values: (Phyma 22.50/22.50, black background): L=79.0;

a=−0.9; b=31.7

### USE EXAMPLES

### Example 1A

### Shower Gel

| | | |
|---|---|---|
| Raw material | INCI | [%] |

A

| | | | |
|---|---|---|---|
| Ronastar ® Golden Sparks | (1) | CALCIUM ALUMINIUM BOROSILICATE, SILICA, | 0.05 |
| | | CI 77891 (TITANIUM DIOXIDE), TIN OXIDE | |
| Multilayer gold pigment | (1) | MICA, CI 77891 (TITANIUM DIOXIDE) Silica, | 0.10 |
| according to Example 4 | | CI 77491 (IRON OXIDES), Alumina, Tin Oxide | |
| Keltrol CG-SFT | (2) | XANTHAN GUM | 1.10 |
| Water, demineralised | | WATER, AQUA (WATER) | 54.90 |

B

| | | | |
|---|---|---|---|
| Plantacare 2000 UP | (3) | DECYL GLUCOSIDE | 20.00 |
| Texapon ASV 50 | (3) | SODIUM LAURETH SULFATE, SODIUM LAURETH-8, | 3.60 |
| | | SULFATE, MAGNESIUM LAURETH SULFATE, | |
| | | MAGNESIUM LAURETH-8 SULFATE, SODIUM | |
| | | OLETH, SULFATE, MAGNESIUM OLETH SULFATE | |
| Bronidox L | (3) | PROPYLENE GLYCOL | 0.30 |
| | | 5-BROMO-5-NITRO-1,3-DIOXANE | |
| Frag 280851 Fruit Cocktail | (4) | PARFUM | 0.20 |
| 0.1% of Sicovit Quinoline | (5) | AQUA (WATER), WATER, CI 47005 | 8.30 |
| Yellow 70 E 104 in water | | (ACID YELLOW 3), ACID YELLOW 3 | |
| 0.1% of Dragocolor True Blue | (6) | AQUA (WATER), WATER, CI 42090 | 1.30 |
| in water | | (FD&C BLUE NO. 1), FD&C BLUE NO. 1 | |

C

| | | | |
|---|---|---|---|
| Citric acid monohydrate | (1) | CITRIC ACID | 0.15 |
| Water, demineralised | | WATER, AQUA (WATER) | 10.00 |

### Preparation:

Phase A: Introduce the water into the reactor and stir in the pigment. Scatter in the Keltrol CG-SFT slowly with stirring and stir until it has completely dissolved (do not homogenise). Add the constituents of phase B individually to phase A. Dissolve the citric acid monohydrate in water and add to the batch, and stir slowly until everything is homogeneously distributed. Adjust the pH to 6.0-6.5 with the addition of citric acid (if necessary).

### Sources of Supply:

### (1) Merck KGaA/Rona®

### (2) C. P. Kelco

### (3) Cognis GmbH

### (4) Drom

### (5) BASF AG

### (6) Symrise

### Example 2A

### Eye Shadow

| | | |
|---|---|---|
| Raw material | INCI | [%] |

A

| | | | |
|---|---|---|---|
| Multilayer gold pigment | (1) | MICA, CI 77891 (TITANIUM DIOXIDE) Silica, | 25.00 |
| according to Example 4 | | CI 77491 (IRON OXIDES), Alumina, Tin Oxide | |
| Timiron ® Splendid Gold | (1) | CI 77891(TITANIUM DIOXIDE), MICA, SILICA | 5.00 |
| Talc | (1) | TALC | 49.50 |
| Potato starch | (2) | POTATO STARCH, SOLANUM TUBEROSUM | 7.50 |
| | | (POTATO STARCH) | |
| Magnesium stearate | (1) | MAGNESIUM STEARATE | 2.50 |

B

| | | | |
|---|---|---|---|
| Isopropyl stearate | (3) | ISOPROPYL STEARATE | 9.34 |
| Cetyl palmitate | (1) | CETYL PALMITATE | 0.53 |
| Ewalin 1751 | (4) | PETROLATUM | 0.53 |
| Perfume oil Elegance + 79228 | (5) | PARFUM | 0.20 |
| D MF | | | |
| Propyl 4-hydroxybenzoate | (1) | PROPYLPARABEN | 0.10 |

### Preparation:

Combine and pre-mix the constituents of phase A. Subsequently add the molten phase B dropwise to the powder mixture with stirring. Introduce the powders into powder pans of large diameter and press at 80 bar.

### Sources of Supply;

### (1) Merck KGaA/Rona®

### (2) Suedstaerke GmbH

### (3) Cognis GmbH

### (4) H. Erhard Wagner GmbH

### (5) Symrise

### Example 3A

### Day Cream (O/W)

| | | |
|---|---|---|
| Raw material | INCI | [%] |

A

| | | | |
|---|---|---|---|
| Ronasphere ® LDP | (1) | SILICA, CI 77891 (TITANIUM DIOXIDE), CI 77491 | 5.00 |
| | | (IRON OXIDES) | |
| Multilayer gold pigment | (1) | MICA, CI 77891 (TITANIUM DIOXIDE) Silica, | 0.10 |
| according to Example 4 | | CI 77491 (IRON OXIDES), Alumina, Tin Oxide | |
| Veegum HV | (2) | MAGNESIUM ALUMINIUM SILICATE | 1.00 |
| Karion F liquid | (1) | SORBITOL | 3.00 |
| Methyl 4-hydroxybenzoate | (1) | METHYLPARABEN | 0.18 |
| Water, demineralised | | AQUA (WATER) | 56.34 |

B

| | | | |
|---|---|---|---|
| Arlacel 165 VP | (3) | GLYCERYL STEARATE, PEG-100 STEARATE | 5.00 |
| Lanette O | (4) | CETEARYL ALCOHOL | 1.50 |
| Miglyol 812 N | (5) | CAPRYLIC/CAPRIC TRIGLYCERIDE | 7.00 |
| Shea butter solid | (6) | BUTYROSPERMUM PARKII (SHEA BUTTER) | 2.00 |
| Cetiol SN | (4) | CETEARYL ISONONANOATE | 7.00 |
| Eutanol G | (4) | OCTYLDODECANOL | 7.50 |
| Emulgade PL 68/50 | (4) | CETEARYL ALCOHOL, CETEARYL GLUCOSIDE | 2.00 |
| Propyl 4-hydroxybenzoate | (1) | PROPYLPARABEN | 0.08 |

C

| | | | |
|---|---|---|---|
| Perfume oil 200 530 | (7) | PARFUM | 0.20 |
| Dow Corning 345 | (8) | CYCLOMETHICONE | 2.00 |
| Euxyl K 400 | (9) | PHENOXYETHANOL, METHYLDIBROMO, | 0.10 |
| | | GLUTARONITRILE | |
| Citric acid monohydrate | (1) | CITRIC ACID | 0.00 |

### Preparation:

Warm phase B until the solution is clear. Disperse the Veegum in the water of phase A, add the remaining raw materials, heat to 80° C. and add phase B. Homogenise phase A/B. Cool to 40° C. with stirring and add phase C. Cool to room temperature and adjust to pH 6.0.

### Sources of Supply:

### (1) Merck KGaA/Rona®

### (2) Vanderbilt

### (3) Uniqema

### (4) Cognis GmbH

### (5) Sasol Germany GmbH

### (6) H. Erhard Wagner GmbH

### (7) Fragrance Resources

### (8) Dow Corning

(9) Schülke & Mayr GmbH

### Example 4A

### Sparkling Body Cream (O/W)

| | | |
|---|---|---|
| Raw material | INCI | [%] |

A

| | | | |
|---|---|---|---|
| Ronastar ® Golden Sparks | (1) | CALCIUM ALUMINIUM BOROSILICATE, SILICA, | 1.00 |
| | | CI 77891(TITANIUM DIOXIDE), TIN OXIDE | |
| Multilayer gold pigment | (1) | MICA, CI 77891(TITANIUM DIOXIDE), SILICA, | 1.00 |
| according to Example 4 | | CI 77491(IRON OXIDES), ALUMINA, TIN OXIDE | |
| Water, demineralised | | WATER, AQUA (WATER) | 40.60 |
| Carbopol Ultrez 21 | (2) | ACRYLATES/C10-30 ALKYL ACRYLATE CROSS- | 0.60 |
| | | POLYMER | |
| Citric acid monohydrate | (1) | CITRIC ACID | 0.00 |

B

| | | | |
|---|---|---|---|
| Water, demineralised | | WATER, AQUA (WATER) | 26.35 |
| 1,2-Propanediol | (1) | PROPYLENE GLYCOL | 3.00 |
| RonaCare ® allantoin | (1) | ALLANTOIN | 0.20 |

C

| | | | |
|---|---|---|---|
| Paraffin liquid | (1) | PARAFFINUM LIQUIDUM (MINERAL OIL), | 10.00 |
| | | MINERAL OIL | |
| Cetiol V | (3) | DECYL OLEATE | 6.00 |
| Hostaphat KL 340 D | (4) | TRILAURETH-4 PHOSPHATE | 3.00 |
| Cetyl alcohol | (1) | CETYL ALCOHOL | 2.00 |
| Phenonip | (5) | PHENOXYETHANOL, BUTYLPARABEN, ETHYL- | 0.50 |
| | | PARABEN, PROPYLPARABEN, METHYLPARABEN | |

D

| | | |
|---|---|---|
| Water, demineralised | WATER, AQUA (WATER) | 3.50 |
| Triethanolamine | TRIETHANOLAMINE | 0.35 |

E

| | | | |
|---|---|---|---|
| Germall 115 | (6) | IMIDAZOLIDINYL UREA | 0.30 |
| Perfume oil Vogue | (7) | PARFUM | 0.10 |
| Water, demineralised | | WATER, AQUA (WATER) | 1.50 |

### Preparation:

Disperse the pearlescent pigment in the water of phase A. If necessary, acidify using a few drops of citric acid in order to reduce the viscosity. Scatter in the Carbopol with stirring. When completely dissolved, slowly stir in the pre-dissolved phase B. Heat phase A/B and phase C to 80° C., stir phase C into phase A/B, homogenise with phase D, neutralise, homogenise again and cool with stirring. Dissolve the Germall 115 in the water of phase E at 40° C., add with stirring. Then add the perfume oil and cool to room temperature with stirring.

### Sources of Supply:

### (1) Merck KGaA/Rona®

### (2) Noveon

### (3) Cognis GmbH

### (4) Clariant GmbH

### (5) Nipa Laboratorien GmbH

### (6) ISP Global Technologies

### (7) Drom

### Example 5A

### Creamy Eye Shadow

| | | |
|---|---|---|
| Raw material | INCI | [%] |

A

| | | | |
|---|---|---|---|
| Multilayer gold pigment | (1) | MICA, CI 77891(TITANIUM DIOXIDE), SILICA, | 20.00 |
| according to Example 4 | | CI 77491(IRON OXIDES), ALUMINA, TIN OXIDE | |
| Micronasphere ® M | (1) | MICA, SILICA | 6.00 |
| Unipure Green LC 789 CF | (2) | CI 77289 (CHROMIUM HYDROXIDE GREEN) | 4.00 |

B

| | | | |
|---|---|---|---|
| Crodamol PMP | (3) | PPG-2 MYRISTYL ETHER PROPIONATE | 37.80 |
| Syncrowax HGLC | (3) | C18-36 ACID TRIGLYCERIDE | 10.00 |
| Syncrowax HRC | (3) | TRIBEHENIN | 3.00 |
| Miglyol 812 N | (4) | CAPRYLIC/CAPRIC TRIGLYCERIDE | 14.00 |
| Stearic acid | (1) | STEARIC ACID | 3.00 |
| Antaron V-216 | (5) | PVP/HEXADECENE COPOLYMER | 2.00 |
| Oxynex ® K liquid | (1) | PEG-8, TOCOPHEROL, ASCORBYL PALMITATE, | 0.10 |
| | | ASCORBIC ACID, CITRIC ACID | |
| Propyl 4-hydroxybenzoate | (1) | PROPYLPARABEN | 0.10 |

### Preparation:

Heat phase B to about 80° C. until everything has melted and cool to 65° C. Then add the pearlescent pigment, Micronasphere and the ground chromium oxide of phase A with stirring. Transfer the eye shadow into containers at 65° C.

### Sources of Supply:

### (1) Merck KGaA/Rona®

### (2) Les Colorants Wackherr

### (3) Croda GmbH

### (4) Sasol Germany GmbH

### (5) ISP Global Technologies

### Example 6A

### Hair Styling Gel

| | | |
|---|---|---|
| Raw material | INCI | [%] |

A

| | | | |
|---|---|---|---|
| Ronastar ® Golden Sparks | (1) | CALCIUM ALUMINIUM BOROSILICATE, CI 77891 | 2.55 |
| | | (TITANIUM DIOXIDE), SILICA, TIN OXIDE | |
| Multilayer gold pigment | (1) | MICA, CI 77891 (TITANIUM DIOXIDE) Silica, | 0.10 |
| according to Example 4 | | CI 77491 (IRON OXIDES), Alumina, Tin Oxide | |
| Carbopol Ultrez 21 | (2) | ACRYLATES/C10-30 ALKYL ACRYLATE CROSS- | 0.90 |
| | | POLYMER | |
| Water, demineralised | | WATER, AQUA (WATER) | 50.35 |

B

| | | | |
|---|---|---|---|
| Luviskol K 30 powder | (3) | PVP | 2.00 |
| Germaben II | (4) | PROPYLENE GLYCOL, DIAZOLIDINYL UREA, | 1.00 |
| | | METHYLPARABEN, PROPYLPARABEN | |
| Triethanolamine, extra pure | (1) | TRIETHANOLAMINE | 2.16 |
| Water, demineralised | | WATER, AQUA (WATER) | 40.94 |

### Preparation:

Disperse the pearlescent pigments in the water of phase A and scatter in the Carbopol with stirring. When completely dissolved, slowly stir in the pre-dissolved phase B.

### Sources of Supply:

### (1) Merck KGaA/Rona®

### (2) Noveon

### (3) BASF AG

### (4) ISP Global Technologies

### Example 7A

### Shampoo

| | | |
|---|---|---|
| Raw material | INCI | [%] |

A

| | | | |
|---|---|---|---|
| Multilayer gold pigment | (1) | MICA, CI 77891(TITANIUM DIOXIDE), SILICA, | 0.20 |
| according to Example 4 | | CI 77491(IRON OXIDES), ALUMINA, TIN OXIDE | |
| Carbopol ETD 2020 | (2) | ACRYLATES/C10-30 ALKYL ACRYLATE CROSS- | 0.90 |
| | | POLYMER | |
| Water, demineralised | | AQUA (WATER) | 63.40 |

B

| | | | |
|---|---|---|---|
| Triethanolamine, extra pure | (1) | TRIETHANOLAMINE | 0.90 |
| Water, demineralised | | AQUA (WATER) | 10.00 |

C

| | | | |
|---|---|---|---|
| Plantacare 2000 UP | (3) | DECYL GLUCOSIDE | 20.00 |
| Texapon ASV 50 | (3) | SODIUM LAURETH SULFATE, SODIUM LAURETH-8 | 4.35 |
| | | SULFATE, MAGNESIUM LAURETH SULFATE, | |
| | | MAGNESIUM LAURETH-8 SULFATE, SODIUM | |
| | | OLETH SULFATE, MAGNESIUM OLETH SULFATE | |
| Bronidox L | (3) | PROPYLENE GLYCOL, 5-BROMO-5-NITRO-1,3-DIOXANE | 0.20 |
| Perfume oil 200 524 | (4) | PARFUM | 0.05 |
| Dye solution (q.s.) | | | 0.00 |

### Preparation:

For phase A, stir the pigment into the water. Acidify using a few drops of citric acid (10%) in order to reduce the viscosity, and slowly scatter in the Carbopol with stirring. When completely dissolved, slowly add phase B. Then add the constituents of phase C successively. Adjust the pH to 6.0-6.5.

### Sources of Supply:

### (1) Merck KGaA/Rona®

### (2) Noveon

### (3) Cognis GmbH

### (4) Fragrance Resources

### Example 8A

### Shimmering Body Powder

| | | |
|---|---|---|
| Raw material | INCI | [%] |

A

| | | | |
|---|---|---|---|
| Multilayer gold pigment | (1) | MICA, CI 77891(TITANIUM DIOXIDE), SILICA, | 10.00 |
| according to Example 4 | | CI 77491(IRON OXIDES), ALUMINA, TIN OXIDE | |

B

| | | | |
|---|---|---|---|
| Microna ® Matte Red | (1) | CI 77491 (IRON OXIDES), MICA | 1.00 |
| Microna ® Matte Yellow | (1) | MICA, CI 77492 (IRON OXIDES) | 1.00 |
| Ronasphere ® LDP | (1) | SILICA, CI 77891 (TITANIUM DIOXIDE), CI 77491 | 4.00 |
| | | (IRON OXIDES) | |
| Talc | (1) | TALC | 25.00 |
| Glass flakes | (1) | CALCIUM ALUMINIUM BOROSILICATE | 15.00 |
| White clay | (1) | KAOLIN | 14.70 |
| Mica M | (1) | MICA | 15.00 |
| Silk mica | (1) | MICA | 9.50 |
| Propyl 4-hydroxybenzoate | (1) | PROPYLPARABEN | 0.30 |

C

| | | | |
|---|---|---|---|
| Cetiol SQ | (2) | SQUALANE | 2.00 |
| Miglyol 812 N | (3) | CAPRYLIC/CAPRIC TRIGLYCERIDE | 2.00 |
| RonaCare ® tocopherol acetate | (1) | TOCOPHERYL ACETATE | 0.20 |
| Perfume | (4) | PARFUM | 0.30 |

### Preparation:

Weigh out all constituents of phase B together and grind homogeneously in a mixer. Subsequently add phase C and continue mixing, then add phase A and grind briefly until the pearlescent pigment is uniformly distributed.

### Sources of Supply:

### (1) Merck KGaA/Rona®

### (2) Cognis GmbH

### (3) Sasol Germany GmbH

### (4) Symrise

### Example 9A

### Long-Lasting Lip Gloss

| | | |
|---|---|---|
| Raw material | INCI | [%] |

A

| | | | |
|---|---|---|---|
| Multilayer gold pigment | (1) | MICA, CI 77891(TITANIUM DIOXIDE), SILICA, | 4.00 |
| according to Example 4 | | CI 77491 (IRON OXIDES), ALUMINA, TIN OXIDE | |
| Ronastar ® Golden Sparks | (1) | CALCIUM ALUMINIUM BOROSILICATE, SILICA, | 6.00 |
| | | CI 77891 (TITANIUM DIOXIDE), TIN OXIDE | |

B

| | | | |
|---|---|---|---|
| Indopol H 100 | (2) | POLYBUTENE | 30.00 |
| Jojoba Glaze LV | (3) | SIMMONDSIA CHINENSIS (JOJOBA), JOJOBA, | 20.00 |
| | | SEED OIL, ETHYLENE/PROPYLENE/STYRENE | |
| | | COPOLYMER, BUTYLENE/ETHYLENE/STYRENE | |
| | | COPOLYMER | |
| Jojoba Glaze HV | (3) | SIMMONDSIA CHINENSIS (JOJOBA), JOJOBA, | 10.00 |
| | | SEED OIL, ETHYLENE/PROPYLENE/STYRENE | |
| | | COPOLYMER, BUTYLENE/ETHYLENE/STYRENE | |
| | | COPOLYMER | |
| Castor oil | (4) | CASTOR OIL, RICINUS COMMUNIS (CASTOR OIL) | 23.15 |
| Beeswax, bleached | (1) | BEESWAX, CERA ALBA (BEESWAX) | 4.00 |
| Propyl 4-hydroxybenzoate | (1) | PROPYLPARABEN | 0.10 |
| Oxynex ® K liquid | (1) | PEG-8, TOCOPHEROL, ASCORBYL PALMITATE, | 0.05 |
| | | ASCORBIC ACID, CITRIC ACID | |
| Jaune Covapate W 1761 | (5) | RICINUS COMMUNIS (CASTOR OIL), CI 19140 | 1.00 |
| | | (FD&C YELLOW No. 5 ALUMINIUM LAKE) | |

C

| | | | |
|---|---|---|---|
| Neosil CT11 | (6) | SILICA | 1.50 |
| Fragrance Tendresse 75418C | (7) | PARFUM | 0.20 |

### Preparation:

Weigh out all constituents of phase B together, heat to 80° C. and stir well. Stir in the pigments of phase A, scatter in the Neosil with stirring, and finally add the perfume. Transfer the homogeneous mixture into containers.

### Sources of Supply:

### (1) Merck KGaA/Rona®

### (2) BP Lavera Sud

### (3) Desert Whale

### (4) Henry Lamotte GmbH

### (5) Les Colorants Wackherr

### (6) Ineos Silicas Limited

### (7) Symrise

### Example 10A

### Nail Varnish

| Raw material | | INCI | [%] |
|---|---|---|---|
| Multilayer gold pigment | (1) | MICA, CI 77891(TITANIUM DIOXIDE), SILICA. | 1.75 |
| according to Example 4 | | CI 77491 (IRON OXIDES), ALUMINA, TIN OXIDE | |
| Ronastar ® Golden Sparks | (1) | CALCIUM ALUMINIUM BOROSILICATE, SILICA, | 0.25 |
| | | CI 77891 (TITANIUM DIOXIDE), TIN OXIDE | |
| Thixotropic nail varnish base | (2) | BUTYL ACETATE, ETHYL ACETATE, NITRO- | 98.00 |
| 155 | | CELLULOSE, ACETYL TRIBUTYL CITRATE, | |
| | | PHTHALIC ANHYDRIDE/TRIMELLITIC | |
| | | ANHYDRIDE/GLYCOLS COPOLYMER, ISOPROPYL | |
| | | ALCOHOL, STEARALKONIUM HECTORITE, ADIPIC | |
| | | ACID/FUMARIC ACID/PHTHALIC | |
| | | ACID/TRICYCLODECANE DIMETHANOL | |
| | | COPOLYMER, CITRIC ACID | |

### Preparation:

Weigh out the pigments together with the varnish base, mix well by hand using a spatula and subsequently stir at 1000 rpm for 10 min.

### Sources of Supply:

### (1) Merck KGaA/Rona®

### (2) Durlin/Bergerac NC

### Example 11A

### Volume Mascara (O/W)

| | | |
|---|---|---|
| Raw material | INCI | [%] |

A

| | | | |
|---|---|---|---|
| Mica Black | (1) | CI 77499 (IRON OXIDES), MICA, CI 77891 | 5.00 |
| | | (TITANIUM DIOXIDE) | |
| Colorona ® Red Brown | (1) | MICA, CI 77491 (IRON OXIDES), CI 77891 | 3.00 |
| | | (TITANIUM DIOXIDE) | |
| Multilayer gold pigment | (1) | MICA, CI 77891 (TITANIUM DIOXIDE) Silica, | 2.00 |
| according to Example 4 | | CI 77491 (IRON OXIDES), Alumina, Tin Oxide | |
| Satin mica | (1) | MICA | 2.00 |

B

| | | | |
|---|---|---|---|
| Dermacryl 79 | (2) | ACRYLATES/OCTYLACRYLAMIDE COPOLYMER | 3.50 |
| Beeswax, bleached | (1) | BEESWAX, CERA ALBA (BEESWAX) | 3.00 |
| Syncrowax HRC | (3) | TRIBEHENIN | 3.50 |
| Stearic acid | (1) | STEARIC ACID | 5.00 |
| Tegin M | (4) | GLYCERYL STEARATE | 3.50 |
| Tegosoft CT | (4) | CAPRYLIC/CAPRIC TRIGLYCERIDE | 2.50 |
| Dow Corning 556 | (5) | PHENYL TRIMETHICONE | 2.00 |
| RonaCare ® tocopherol acetate | (1) | TOCOPHERYL ACETATE | 0.50 |
| Phenonip | (6) | PHENOXYETHANOL, BUTYLPARABEN, ETHYL- | 0.80 |
| | | PARABEN, PROPYLPARABEN, METHYLPARABEN | |

C

| | | | |
|---|---|---|---|
| Water, demineralised | | WATER, AQUA (WATER) | 59.15 |
| AMP Ultra PC 1000 | (7) | AMINOMETHYL PROPANOL | 1.25 |
| 1,3-Butanediol | (1) | BUTYLENE GLYCOL | 1.00 |
| RonaCare ® Biotin Plus | (1) | UREA, DISODIUM PHOSPHATE, BIOTIN, CITRIC | 0.50 |
| | | ACID | |

D

| | | | |
|---|---|---|---|
| Germall 115 | (8) | IMIDAZOLIDINYL UREA | 0.30 |
| Water, demineralised | | WATER, AQUA (WATER) | 1.50 |

### Preparation:

Melt all constituents of phase B, apart from the Dermacryl 79, together at about 85° C., add the Dermacryl 79 with stirring, and stir for 20 min until everything is homogeneously distributed. Heat the constituents of phase C to about 85° C. Stir the pearlescent pigments of phase A into phase C. Add phase C to phase B, continue stirring, and homogenise at 8000 rpm using the Ultra-Turrax T25 for 1 min. Allow to cool with stirring, and add phase D at 40° C.

### Sources of Supply:

### (1) Merck KGaA/Rona®

### (2) National Starch & Chemical

### (3) Croda GmbH

### (4) Degussa-Goldschmidt AG

### (5) Dow Corning

### (6) Nipa Laboratorien GmbH

### (7) Angus Chemie GmbH

### (8) ISP Global Technologies

### Example 12A

### Tinted Day Cream with UV Protection (O/W)

| | | |
|---|---|---|
| Raw material | INCI | [%] |

A

| | | | |
|---|---|---|---|
| Eusolex ® 2292 | (1) | ETHYLHEXYL METHOXYCINNAMATE, BHT | 3.00 |
| Eusolex ® 4360 | (1) | BENZOPHENONE-3 | 3.00 |
| Arlacel 165 VP | (2) | GLYCERYL STEARATE, PEG-100 STEARATE | 5.00 |
| Eusolex ® HMS | (1) | HOMOSALATE | 5.00 |
| Arlacel 165 VP | (2) | GLYCERYL STEARATE, PEG-100 STEARATE | 3.00 |
| Montanov 68 | (3) | CETEARYL ALCOHOL, CETEARYL GLUCOSIDE | 3.00 |
| Dow Corning 345 | (4) | CYCLOMETHICONE | 0.50 |
| Eutanol G | (5) | OCTYLDODECANOL | 2.00 |
| Propyl 4-hydroxybenzoate | (1) | PROPYLPARABEN | 0.05 |

B

| | | | |
|---|---|---|---|
| Eusolex ® T-2000 | (1) | TITANIUM DIOXIDE, ALUMINA, SIMETHICONE | 3.00 |
| Extender W | (1) | MICA, CI 77891 (TITANIUM DIOXIDE) | 4.00 |
| Microna ® Matte Yellow | (1) | MICA, CI 77492 (IRON OXIDES) | 2.00 |
| Microna ® Matte Orange | (1) | MICA, CI 77491 (IRON OXIDES) | 0.20 |
| Microna ® Matte Red | (1) | CI 77491 (IRON OXIDES), MICA | 0.20 |
| Microna ® Matte Black | (1) | CI 77499 (IRON OXIDES), MICA | 0.20 |
| Multilayer gold pigment | (1) | MICA, CI 77891 (TITANIUM DIOXIDE) Silica, | 2.00 |
| according to Example 4 | | CI 77491 (IRON OXIDES), Alumina, Tin Oxide | |
| Karion FP, liquid | (1) | SORBITOL | 5.00 |
| RonaCare ® allantoin | (1) | ALLANTOIN | 0.50 |
| Keltrol T | (6) | XANTHAN GUM | 0.20 |
| Chemag 2000 | (7) | IMIDAZOLIDINYL UREA | 0.30 |
| Euxyl K 400 | (8) | PHENOXYETHANOL, METHYLDIBROMO | 0.10 |
| | | GLUTARONITRILE | |
| Methyl 4-hydroxybenzoate | (1) | METHYLPARABEN | 0.15 |
| Water, demineralised | | AQUA (WATER) | 57.60 |

### Preparation:

Disperse all constituents, apart from the Keltrol T, in the water of phase B. Scatter the Keltrol into phase B with stirring, and, after 15 minutes, heat to 80° C. Heat phase A to 75° C. Slowly stir phase B into phase A and homogenise. Cool with stirring.

### Sources of Supply:

### (1) Merck KGaA/Rona®

### (2) Uniqema

### (3) Seppic

### (4) Dow Corning

### (5) Cognis GmbH

### (6) C. P. Kelco

### (7) Chemag AG

(8) Schülke & Mayr GmbH

### Example 13A

### Cream Conditioner

| Phase | Raw material | | INCI | % |
|---|---|---|---|---|
| A | Water | | Aqua (water) | 79.7 |
| | Multilayer gold | (1) | MICA, CI 77891 (TITANIUM | 0.50 |
| | pigment according | | DIOXIDE) Silica, CI 77491 | |
| | to Example 4 | | (IRON OXIDES), Alumina, | |
| | | | Tin Oxide | |
| | Luviquat Hold | (2) | Polyquaternium-46 | 5.00 |
| | Luviquat PQ 11 | (2) | Polyquaternium-11 | 2.00 |
| | 1,3-Butanediol | (1) | Butylene Glycol | 3.00 |
| B | Cremophor A 6 | (2) | Ceteareth-6 and Stearyl | 3.00 |
| | | | Alcohol | |
| | Ammonyx 4 | (2) | Stearalkonium chloride | 3.00 |
| | Lanette Wax O | (3) | Cetearyl Alcohol | 2.00 |
| | Eusolex 2292 | (1) | Octyl Methoxycinnamate | 0.10 |
| C | RonaCare ® | (1) | Tocopheryl acetate | 0.50 |
| | tocopherol | | | |
| | acetate | | | |
| | RonaCare ® | (1) | Bisabolol | 0.10 |
| | bisabolol nat. | | | |
| | Perfume | | Parfum | 0.10 |
| | Germaben II | (4) | Propylene glycol, | 1.00 |
| | | | Diazolidinyl urea, | |
| | | | Methylparaben, | |
| | | | Propylparaben | |

### Preparation:

Disperse the pigments in the water of phase A, and add the remaining raw materials. Stir after each addition and subsequently heat to 75° C. Mix the raw materials of phase B, heat to 75-80° C. and add to phase A. Mix until a homogeneous distribution is present. Add phase C at 45° C.

### Sources of Supply:

### (1) Merck KGaA/Rona®

### (2) BASF AG

### (3) Cognis GmbH

### (4) ISP Global Technologies

The cosmetic formulations of Examples 1A-13A are distinguished by their intense golden lustre and their very good skin feel.

### Example 14A

### Production of Hard Caramels

| Raw material | % | Sources of supply: |
|---|---|---|
| Sugar | 41% | Südzucker |
| Water | 17.118% | |
| Glucose syrup | 41% C* Sweet | Cerestar, Krefeld |
| Multilayer gold | 0.082% (0.1% based on | Merck KGaA, |
| pigment according | the pouring material) | Darmstadt |
| to Example 1 | | |
| E 104 1:100 dil. | 0.4% of Sikovit | BASF, Ludwigshafen |
| Aroma | 0.4% (banana | Dragaco, Holzminden |
| | 9/030388) | |

The sugar is heated to 100° C. with the water, and the glucose syrup is then added. The solution is subsequently heated to 145° C. After addition of the gold pigment, the colouring solution and the aroma, the caramel solution is poured into greased moulds using a pouring funnel. Finally, the caramels are allowed to cool for two hours. The gold pigment can be added either mixed with the sugar or mixed with the glucose syrup. This variant contains no acid since this would make the caramelisation too strong.

### Example 15A

### Coating of Tablets

a) Initial weight 1 kg of white tablets d=8 mm, G=200 mg

Solution for film coating:

| | | |
|---|---|---|
| 6% | of Sepifilm Lp10 | Seppic |
| | (mixture of hydroxypropylmethyl- | |
| | cellulose, stearic acid and micro- | |
| | crystalline cellulose) | |
| 5% | of multilayer gold pigment | Merck KGaA, Darmstadt |
| | according to Example 2 | |
| 89% | of water | |

Total application amount: 200 g
This corresponds to 1.2 mg of polymer/cm² of tablet surface

### Preparation of the Film-Coating Solution:

Stir the gold pigment into the water. Subsequently add additional dyes. Finally, scatter the film former (HPMC) into the suspension. Due to the increasing viscosity, the stirring speed must also be increased correspondingly. After about 40-60 minutes, the HPMC has completely dissolved and the solution can then be sprayed onto the tablets.
The spray application is carried out by means of standard coating methods.

1. Pigment mixture consisting of at least two components A and B, characterised in that
component A comprises effect pigments based on multicoated flake-form substrates which have a layer sequence comprising
(A) a high-refractive-index coating consisting of a mixture of TiO₂ and Fe₂O₃ in the molar ratio 1:0.5 to 1:2.0 and optionally one or more metal oxide(s) in amounts of <20% by weight, based on layer (A),
(B) a colourless coating having a refractive index n<1.8,
(C) a colourless coating having a refractive index n>1.8,
(D) an absorbent coating having a refractive index n>1.8
and optionally
(E) an outer protective layer,
and
component B comprises colorants selected from the group of inorganic pigments, organic pigments, dyes, colouring natural fruit and/or plant extracts and/or fillers which consist of flake-form, needle-shaped, spherical or irregularly shaped particles.

2. Pigment mixture according to claim 1, characterised in that the effect pigment of component A has the following layer structure:
substrate+Fe₂O₃/TiO₂+SiO₂+TiO₂+Fe₂O₃/TiO₂
substrate+Fe₂O₃/TiO₂+SiO₂+TiO₂+Fe₂O₃
substrate+Fe₂O₃/TiO₂+SiO₂+TiO₂+SiO₂+Fe₂O₃/TiO₂.

3. Pigment mixture according to claim 1, characterised in that the flake-form substrate of component A is covered with a layer of TiO₂ or SiO₂.

4. Pigment mixture according to claim 1, characterised in that the effect pigment of component A has the following layer structure:
substrate+TiO₂+Fe₂O₃/TiO₂+SiO₂+TiO₂+Fe₂O₃/TiO₂
substrate+TiO₂+Fe₂O₃/TiO₂+SiO₂+TiO₂+Fe₂O₃.

5. Pigment mixture according to claim 1, characterised in that the molar ratio of TiO₂ and Fe₂O₃ of layer (A) of the effect pigment of component A is 1:1.

6. Pigment mixture according to claim 1, characterised in that the molar ratio of TiO₂ and Fe₂O₃ of layer (D) of the effect pigment of component A is 1:1.

7. Pigment mixture according to claim 1, characterised in that layer (A) of the effect pigment of component A is a layer of pseudobrookite or a mixture of pseudobrookite with TiO₂ or pseudobrookite with Fe₂O₃.

8. Pigment mixture according to claim 1, characterised in that layer (D) of the effect pigment of component A is a layer of pseudobrookite or a mixture of pseudobrookite with TiO₂ or pseudobrookite with Fe₂O₃.

9. Pigment mixture according to claim 1, characterised in that layers (A) and (D) of the effect pigment of component A are each a layer of pseudobrookite or a mixture of pseudobrookite with TiO₂ or pseudobrookite with Fe₂O₃.

10. Pigment mixture according to claim 1, characterised in that the colorant of component B is a pearlescent pigment, a multilayer pigment and/or an interference pigment or a mixture thereof which is different from component A.

11. Pigment mixture according to claim 1, characterised in that component A and component B are mixed in the mixing ratio 99:1 to 50:50.

12-17. (canceled)

18. Use of the pigment mixture according to claim 1, in food and pharmaceutical products, characterised in that it is employed in combination with aroma substances and/or sweeteners.

19. Formulations comprising a pigment mixture according to claim 1.

20. Formulations comprising the pigment mixture according to claim 1 in amounts of 0.01-99% by weight, based on the formulation as a whole.

21. Formulations according to claim 19, characterised in that it additionally comprises water, polyols, polar and non-polar oils, fats, waxes, film formers, polymers, copolymers, surfactants, free-radical scavengers, antioxidants, stabilisers, odour enhancers, silicone oils, emulsifiers, solvents, preservatives, thickeners, rheological additives, fragrances, UV absorbers, surface-active assistants and/or cosmetic active compounds.

22. A method comprising incorporating a pigment mixture into a cosmetic formulation, a food, or a pharmaceutical product wherein the pigment mixture comprises components A and B in a mixing ratio of 99:1 to 1:99, wherein
component A comprises effect pigments based on multicoated flake-form substrates which have a layer sequence comprising
(A) a high-refractive-index coating consisting of a mixture of TiO₂ and Fe₂O₃ in the molar ratio 1:0.5 to 1:2.0 and optionally one or more metal oxide(s) in amounts of <20% by weight, based on layer (A),
(B) a colourless coating having a refractive index n<1.8,
(C) a colourless coating having a refractive index n>1.8,
(D) an absorbent coating having a refractive index n>1.8
and optionally
(E) an outer protective layer,
and
component B comprises fillers which consist of flake-form, needle-shaped, spherical or irregularly shaped particles.

23. The method of claim 22 wherein the effect pigment of component A has the following layer structure:
substrate+Fe₂O₃/TiO₂+SiO₂+TiO₂+Fe₂O₃/TiO₂
substrate+Fe₂O₃/TiO₂+SiO₂+TiO₂+Fe₂O₃
substrate+Fe₂O₃/TiO₂+SiO₂+TiO₂+SiO₂+Fe₂O₃/TiO₂.

24. The method of claim 22 wherein the flake-form substrate is covered with a layer of TiO₂ or SiO₂.

25. The method of claim 22 wherein the effect pigment of component A consist of:
substrate+TiO₂+Fe₂O₃/TiO₂+SiO₂+TiO₂+Fe₂O₃/TiO₂
substrate+TiO₂+Fe₂O₃/TiO₂+SiO₂+TiO₂+Fe₂O₃.

26. The method of claim 22 wherein the molar ratio of TiO₂ and Fe₂O₃ of layer (A) of the effect pigment of component A is 1:1.

27. The method of claim 22 wherein the molar ratio of TiO₂ and Fe₂O₃ of layer (D) of the effect pigment of component A is 1:1.

28. The method of claim 22 wherein layer (A) of the effect pigment of component A is a layer of pseudobrookite or a mixture of pseudobrookite with TiO₂ or pseudobrookite with Fe₂O₃.

29. The method of claim 22 wherein layer (D) of the effect pigment of component A is a layer of pseudobrookite or a mixture of pseudobrookite with TiO₂ or pseudobrookite with Fe₂O₃.

30. The method of claim 22 wherein layers (A) and (D) of the effect pigment of component A are each a layer of pseudobrookite or a mixture of pseudobrookite with TiO₂ or pseudobrookite with Fe₂O₃.

31. The method of claim 1 wherein the colorant of component B is a pearlescent pigment, a multilayer pigment and/or an interference pigment or a mixture thereof which is different from component A.

32. The method of claim 22 wherein component A and component B are mixed in the mixing ratio 99:1 to 50:50.

33. The method of claim 22 wherein the pigment mixtures are further combined with aroma substances and/or sweeteners.

34. The method of claim 22 wherein the combination of components A and B comprise 0.01-99% by weight, of the formulation as a whole.
